(19) United States
(12) Patent Application Publication
(10) Pub. No.: US 2023/0214784 A1
(43) Pub. Date: Jul. 6, 2023

(54) SYSTEMS AND METHODS FOR A PREDICTIVE DOUBLE-BOOKING MEDICAL APPOINTMENT SYSTEM

(71) Applicant: Dignity Health, San Francisco, CA (US)

(72) Inventors: Sunilkumar Kakade, San Francisco, CA (US); Umar Iqbal, San Francisco, CA (US); Mark Page, San Francisco, CA (US); Jordan Durham, San Francisco, CA (US); Nilesh Mehta, San Francisco, CA (US); Harry Schned, San Francisco, CA (US); Daniel Hagen, San Francisco, CA (US)

(21) Appl. No.: 17/998,884

(22) PCT Filed: May 18, 2021
(86) PCT No.: PCT/US21/32942

§ 371 Date: Nov. 15, 2022

Related U.S. Application Data
(60) Provisional application No. 63/026,532, filed on May 18, 2020.

(51) Int. Cl. G06Q 10/1093; G16H 40/20

(52) U.S. Cl. CPC G06Q 10/1095; G16H 40/20

(57) ABSTRACT

Various embodiments of a predictive double-booking system for use in medical appointment booking applications are described herein.

### FIELD

The present disclosure generally relates to predictive models; and in particular, to computer-implemented predictive models for determining risk of a patient failing to attend a medical appointment and mitigating lost resources due to patients failing to attend appointments.

### BACKGROUND

No-shows, in which a patient has scheduled an appointment, subsequently failed to attend the appointment, or not given appropriate notice, are pervasive problems that hinder provider and clinic productivity. Because the patients are missing unexpectedly, there is little chance that the slot for such appointment can be recovered and filled by other patients. Reduction interventions, defined as an intervention style which aims to simply reduce an amount of no-shows, would attempt to guide an already-scheduled patient away from being a no-show. An example reduction intervention would be calling the patient to remind them of their upcoming appointment. The intended consequence of such an intervention method would be to either encourage the patient to actually arrive or to cancel/reschedule the appointment.

No-shows result in forfeiting a revenue-generating slot for a given healthcare provider. When considered over a whole enterprise, these lost slots can result in substantial loss of revenue. Patients also can sit idly waiting for days, weeks or even months for a chance to see their provider, which can be demoralizing to the patient and prevent them from receiving necessary care in a timely manner. One solution to the aforementioned problem would be to give unused no-show appointment slots to patients who are waiting for an appointment.

With conventional technology, automated reminder systems can be scaled and distributed to patients for appointment reminders, regardless of their predisposition to no-showing. However, such general automated reminder systems lack sufficient technical features for identifying no-shows despite an appointment. Technical improvements are needed.

It is with these observations in mind, among others, that various aspects of the present disclosure were conceived and developed.

### BRIEF DESCRIPTION OF THE DRAWINGS

FIG. 1 is a simplified block diagram showing an example environment for a predictive double booking system;

FIG. 2 is a simplified block diagram showing components of the predictive double booking system of FIG. 1;

FIG. 3 is a simplified diagram showing data flow between components of the predictive double booking system of FIG. 1;

FIG. 4 is an illustration showing determination of a cumulative risk score based on individual slot risk scores as determined by the predictive double booking system of FIG. 1;

FIG. 5 is a diagram showing one particular implementation of the predictive double booking system of FIG. 1 with respect to various EHR systems;

FIG. 6 is an illustration showing an example user interface view showing a risk summary for a plurality of appointment slots of the predictive double booking system of FIG. 1;

FIG. 7 is an illustration showing an example user interface view showing a provider schedule view with actionable opportunity for double-booking for a plurality of appointment slots using the predictive double booking system of FIG. 1;

FIG. 8 is a graphical representation showing performance metrics over the course of a week using the predictive double booking system of FIG. 1;

FIG. 9 is a flowchart showing one example method executed by the predictive double booking system of FIG. 1; and

FIG. 10 is an example device for implementation of the of the predictive double booking system of FIG. 2.

Corresponding reference characters indicate corresponding elements among the view of the drawings. The headings used in the figures do not limit the scope of the claims.

### DETAILED DESCRIPTION

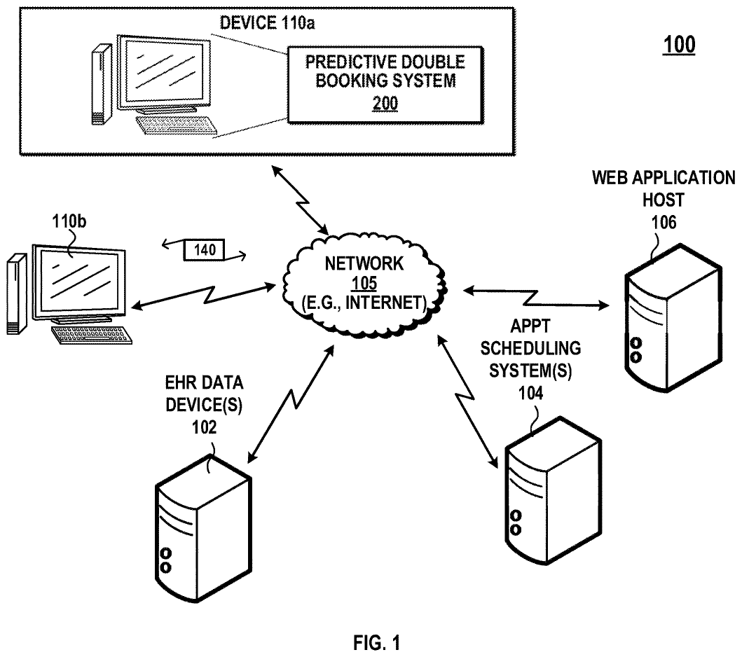

Various embodiments of a computer-implemented system and method which leverages patient demographics, appointment data, and clinic data to determine a risk score representative of a patient failing to attend an appointment are disclosed herein. In one embodiment, a determined risk score for a partial-day of appointments may be used to “double-book” one or more appointment slots based on the determined risk score of the partial day. In some embodiments, each determined risk score is stored in a lookup table, where it is referenced by a reporting tool when relevant. In some embodiments, the reporting tool may be embodied as a scheduling tool, or may otherwise be in communication with the scheduling tool such that a practitioner can access appointment booking information including eligible “double-booking” slots. Referring to the drawings, embodiments of the predictive double-booking platform and related methods and devices are illustrated and generally indicated as 100-500 in FIGS. 1-10.

Referring to the figures, FIG. 1 illustrates a schematic diagram of a predictive double-booking platform 100, which includes an example communication network 105 (e.g., the Internet). Communication network 105 is shown for purposes of illustration and can represent various types of networks, including local area networks (LANs), wide area networks (WANs), telecommunication networks (e.g., 4G, 5G, etc.), and so on.

As shown, communication network 105 includes a geographically distributed collection of client devices, such as devices 110a and 110b (collectively, “devices 110”) along with at least one electronic health record (EHR) data device 102, at least one appointment scheduling system 104 and a web application host 106. Devices 110 and (EHR) data device 102, appointment scheduling system 104 and web application host 106 are interconnected by communication links and/or network segments and exchange or transport data such as data packets 140 to/from a predictive double-booking system 120. Here, devices 110 include main computing device 110a and a client device 110b. Further, devices 110 can include data processing clusters such as a Hadoop cluster. The illustrated client devices represent specific types of electronic devices, but it is appreciated that devices 110 in the broader sense are not limited to such specific devices. For example, devices 110 can include any number of electronic devices such as desktop computers, laptops, smart watches, wearable smart devices, personal digital assistants (PDAs), smart glasses, smart home devices, accessibility devices, other wearables, and so on. In addition, those skilled in the art will understand that any number of devices and links may be used in communication network 105, and that the views shown by FIG. 1 are for simplicity and discussion.

Data packets 140 represent network traffic or messages, which are exchanged between devices 110 and a predictive double-booking system 200 over communication network 105 using predefined network communication protocols such as wired protocols, wireless protocols (e.g., IEEE Std. 802.x, WiFi, Bluetooth®, etc.), Power Line Carrier (PLC) protocols, or other shared-media protocols where appropriate. In this context, a protocol includes a set of rules defining how devices interact with each other.

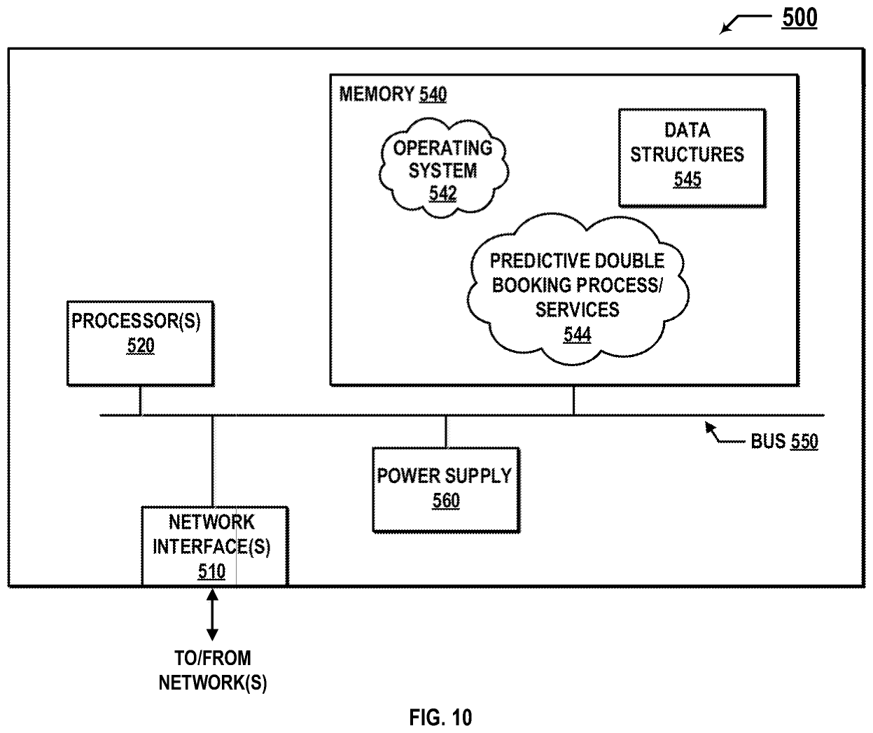

The predictive double-booking system 200, further described herein, may be implemented by one or more processors, processing elements, or devices such as a server, computing device, laptop, desktop computer, and the like; or may be implemented via a cloud or other such hosting environments. In some embodiments, aspects of the system 200 may be embodied as code and/or machine-executable instructions executable by some processor (e.g. processor 520), that may represent one or more of a procedure, a function, a subprogram, a program, a routine, a subroutine, a module, an object, a software package, a class, or any combination of instructions, data structures, or program statements, and the like. In other words, aspects of the system 200 functionality described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) of the instructions may be stored in a computer-readable or machine-readable medium (e.g., memory 540 of FIG. 10), and a processor performs the tasks defined by the code.

The techniques described herein provide a predictive double booking platform that allows providers to assess a risk of at least one patient not attending an appointment given a scheduling interval and various features of the patient and the appointment. In some embodiments, the double booking platform receives attributes associated with an appointment slot within a time interval such as a half-day or quarter-day block. The predictive double booking platform includes a predictive model engine that assesses a risk score of an individual slot being a “no-show”based on the received attributes associated with the slot. In the present system, a slot being at high risk for a “no-show” indicates a high probability that a patient assigned to the slot will not attend the appointment. In some embodiments of the present system, a no-show is defined as an appointment that was classified directly as a no-show in a source system or an appointment that was cancelled on the same calendar day as the scheduled appointment time. Individual slot risks are aggregated across the time interval to provide a cumulative probability that there will be at least one “no-show” among all appointment slots within the time interval. Aggregated risk predictions are timestamped stored in a score lookup table that can be referenced by a scheduling tool or application to generate or manage scheduling reports. Upon identification and subsequent flagging of a significant “no-show” risk within a scheduling block, a provider can offer a double-booked appointment opportunity for an additional consenting patient to be seen sometime within flagged scheduling block. The predictive double booking platform can include or be embodied as various systems (e.g., predictive double-booking system 200), devices (e.g., device 500), and processes (e.g., procedure 400).

### The Problem and Theory of Solution

As noted above, the prevalent nature of modern electronic devices in all aspects of life presents opportunities to create, integrate, and design new technologies for patient appointment booking that enable time recoupment for missed appointments. Mitigation interventions attempt to alleviate the effect of no-shows in medical appointments through indirect means—the goal is not to ensure that the original patient arrives for their appointment but to recoup the loss that results once the patient does not appear for their appointment.

In the predictive double-booking platform 100, a high-risk slot or schedule is double-booked to intentionally book an additional patient on an already-full schedule and filled appointments with the expectation that one of those scheduled patients will be a no-show and not arrive for their appointment. The predictive double-booking platform 100 quantifies how likely it would be to have at least one patient not arrive. Those schedules (or slots, depending on model capability) would then be identified as high-risk and targeted for a potential double-booking opportunity. The patient being double-booked would be fully aware of their status and with their permission would need to opt-in to be scheduled in that manner.

In one embodiment of the predictive double-booking platform 100, schedules are targeted at the healthcare provider half-day level. If the predictive double-booking platform 100 predicts that a provider has a morning or afternoon block of appointments that, collectively, would have a high likelihood of at least one no-show amongst them, then the predictive double-booking platform 100 would recommend that a double-book be scheduled over the high-risk block. Ideally, the recommendation would be as time specific as possible—a patient could end up waiting a long time over the half-day, so it would be beneficial to them (and to the schedulers) to be more precise on when the patient should arrive. In one aspect, the wait time itself could also effectively be predicted and communicated.

### System Overview:

The present predictive double-booking system 200 can include or at least partially be described as a supervised learning model which predicts the risk of a particular appointment being a no-show. These appointment-level risk scores are then aggregated to a provider half-day (or other partial-day segment) level to provide a schedule risk for at least one no-show. A number of no-shows on the schedule could also be predicted via aggregation of individual risk.

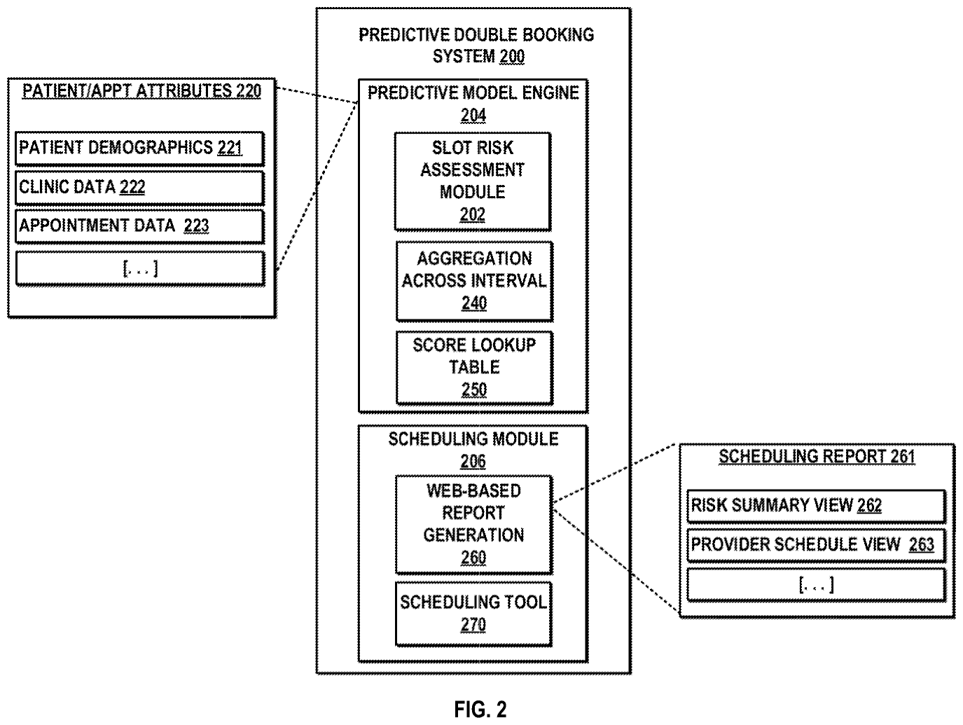

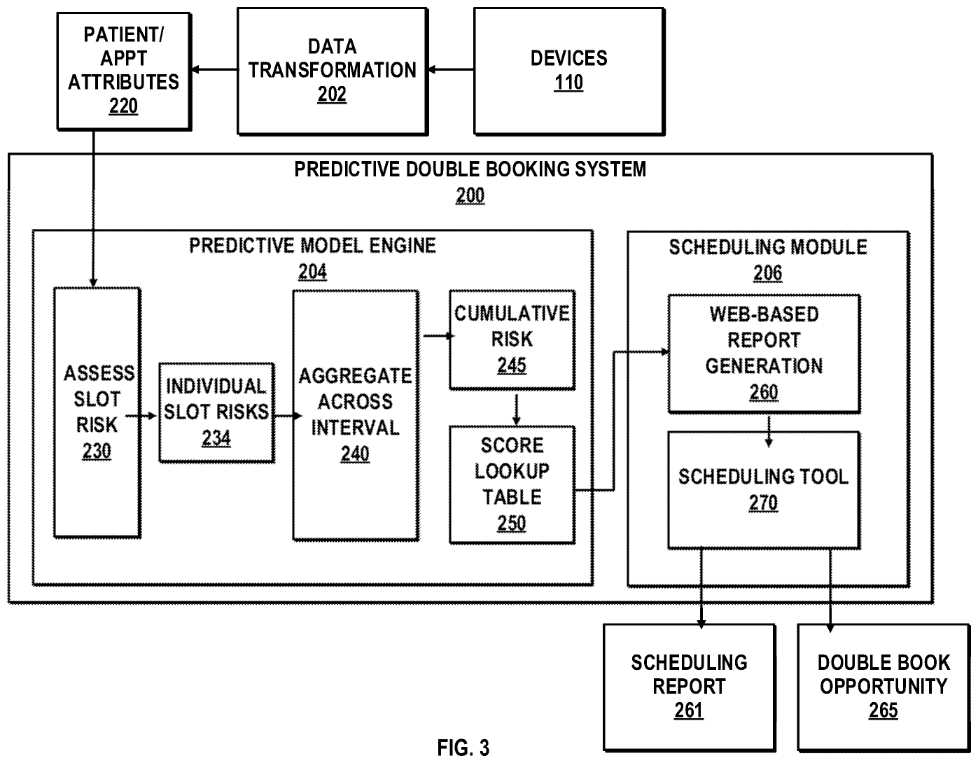

Referring to FIGS. 2 and 3, a first embodiment of predictive double-booking system 200 is shown. The predictive double-booking system 200 includes a predictive model engine 204 and associated scheduling module 206. Predictive model engine 204 determines a no-show risk for a plurality of appointment slots across an interval based on a plurality of attributes 220 related to the appointment and patient. Scheduling module 206 provides scheduling and reporting tools for managing appointment scheduling in view of the no-show risk provided by the predictive model engine 204. In some embodiments, the predictive double booking system 200 includes or otherwise communicates with a data transformation module 202 for cleaning and standardization of attributes 220 as received from the connected devices 110 to ensure that predictive features are interpreted properly.

Referring to FIGS. 2 and 3, the predictive model engine receives a plurality of patient/appointment attributes 220 that are selected as indicative of a potential no-show. Such attributes can include patient attributes and data 221, clinic data 222 and appointment data 223 for each appointment-level slot within a time interval. A slot risk assessment module 230 of predictive model engine 204 predicts appointment-level no-show risks for each individual appointment slot of a plurality of appointment slots through application of a learning technique to the plurality of patient/appointment attributes 220. In some embodiments, this learning technique is a logistic regression model that models a probability that an appointment time slot of a time interval will be classified as a no-show based on risk factors associated with attributes 220. Aggregation module 240 aggregates the appointment-level risk scores to partial-day level corresponding with the time interval (in some embodiments, the time interval is a half-day) through application of probability logic. In some embodiments, slot risk assessment module 230 and aggregation module 240 are implemented on device 500 (FIG. 10) or another suitable device using a master script which takes as input a combination of patient data 221, clinic data 222, and/or appointment data 223. In some embodiments, the aggregated appointment-level risk scores as determined by aggregation module 240 are timestamped and stored in a lookup table 250, which is referenced by a web-based or standalone application report generation tool 260 to generate scheduling reports 261. The scheduling module 206 further displays a double-book opportunity 265 for the time interval with respect to the cumulative risk 245 across the time interval. In some embodiments, the scheduling module 206 or predictive model engine 204 identifies the double-book opportunity 265 by comparing the cumulative risk value 245 with a predetermined threshold value. In some embodiments, the predetermined threshold value is empirically selected.

In some embodiments, a scheduling tool 270 references this scheduling report 261 or lookup table 250; however, in other embodiments the scheduling reports 261 may use their own scheduling tool 270. The predictive double-booking system 200 enables schedulers to quickly and reliably identify opportunities for double-booking a patient over a provider's partial-day or full-day schedule. In some embodiments, high-risk appointments are specifically flagged, providing some extra guidance for when the double-book patient should arrive. Performance of the model can be tracked both visually and statistically.

### Implementation Details

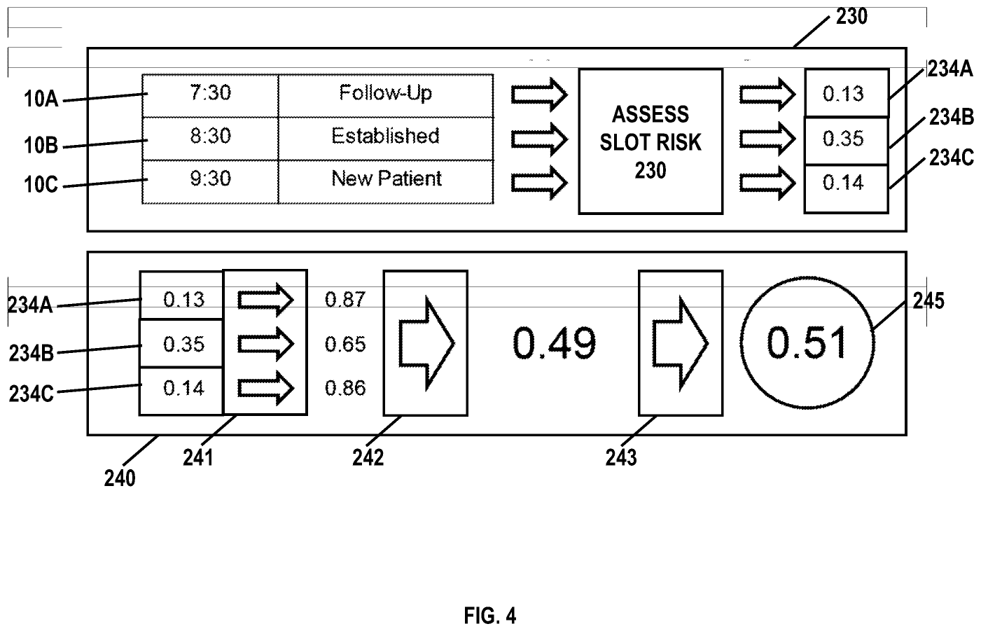

The implementation details may vary between embodiments. Referring to FIGS. 2-4, at the predictive model engine 204, the slot risk assessment module 230 determines appointment-level risk scores based on the attributes 220 using logistic regression or another probability-based learning or modeling technique. Other embodiments of slot risk assessment module 230 may use a gradient boosted decision tree algorithm (embodied as the XgBoost package in Python) to determine appointment-level risk scores. In one embodiment, a single decision tree was built for testing the concept which was pruned to a single, length-3 branch. If all rules in the branch are true based on the attributes 220, an appointment is flagged as high-risk and a historical no-show rate for appointments associated with the rules that were met is assigned as the risk probability for that slot. Otherwise, the appointment is considered low-risk and a prior no-show rate for appointments associated with not meeting all the rules is assigned as the risk probability. Slot risk assessment module 230 returns individual slot risks 234 for each appointment time slot of the plurality of appointment time slots. In an example shown in FIG. 4, three appointment time slots 10A-10C are shown respectively associated with individual slot risks 234A-C. In some embodiments, slot risk assessment module 230 is implemented in Structured Query Language (SQL).

At aggregation module 240, the individual slot risks 234 for each appointment are aggregated across a time interval. As discussed above, one example time interval is a half-day, but other increments such as a shift length, quarter day, and full day can be selected. Aggregation module 240 aggregates individual slot risks 234 across the time interval using binomial probability logic, a process illustrated in FIG. 4. The probability of there being at least one no-show in a given time interval is equal to one minus the probability that there will be zero no-shows in the time interval (P(1+)=1−P(0)). Because each appointment is considered to be statistically independent from one another, at block 241 the aggregation module 240 takes one minus the individual slot risk 234 to retrieve an arrival probability of each patient arriving for their respective appointment. At block 242 the aggregation module 240 multiplies each of the arrival probabilities together to obtain a probability that all patients in the time interval will arrive. In the example shown, individual slot risks 234A-C respectively associated with appointment time slots 10A-C are multiplied together. This is equivalent to the probability that no patient no-shows during the time interval. Taking one minus the probability that no patient no-shows during the time interval as illustrated at block 243 yields a cumulative risk 245 that at least one patient will no-show and that at least one appointment time slot across the time interval will be classified as a “no-show”.

In some embodiments, a master script for predictive model engine 204 is built using Structured Query Language (SQL) and runs each of the intermediate parts, including data transformation module 202 to ensure that predictive features are interpreted properly from the patient data 221, clinic data 222 and appointment data 223, clinic classification scoring, universal scoring, and time interval aggregation and distribution. The aggregated predictions are timestamped and stored in a lookup table 250 to be referenced by scheduling module 206 of the predictive double booking system 200.

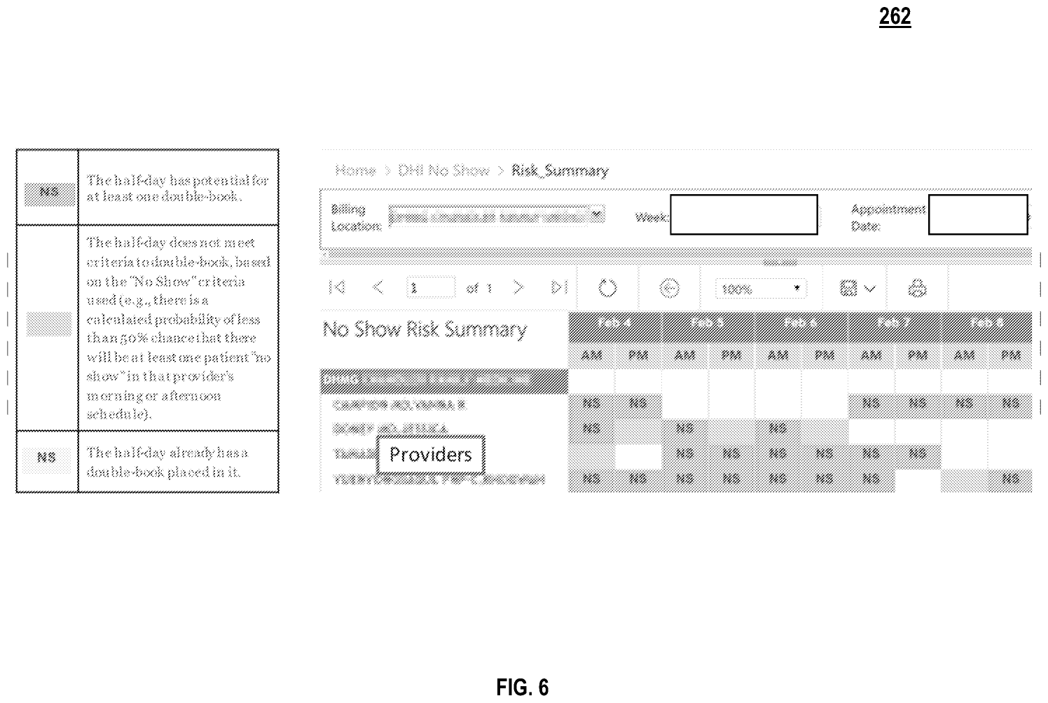

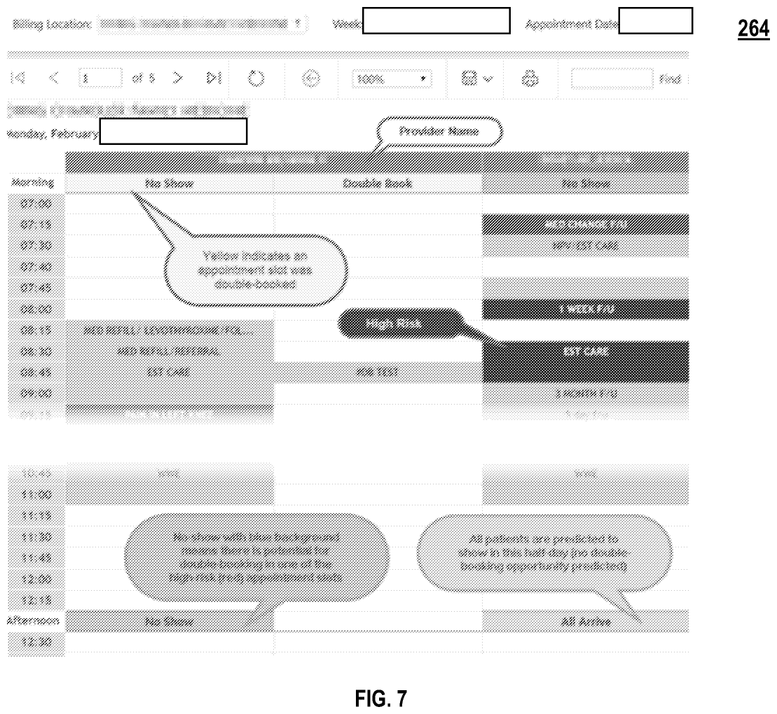

In some embodiments, the scheduling module communicates with the lookup table 250 to generate scheduling report 261 at a report generation module 260. In some embodiments, the scheduling module 206 or aspects of the scheduling module 206 are web-based and can be implemented through a web application host such as Microsoft SQL. The risk scores are translated into actionable statements of double-book opportunity using a scheduling tool 270. In some embodiments, the model itself is stored in a table and parsed using a stored procedure. The scheduling report 261 can include various graphic user interface (GUI) views including a risk summary view 262 (shown in FIG. 6) and a provider schedule view 263 (shown in FIG. 7). The scheduling module 206 allows a user to double-book an additional appointment time interval based on the risk score for the time interval as determined by the predictive model engine 204. In some embodiments, schedulers will operationally decide whether to double-book a patient over a time interval after viewing associated risk scores for the time interval, typically in a passive fashion. For instance, consider the patient called in to schedule an appointment. The scheduler views the scheduling report 261 to identify if a double-book opportunity is available for the requested provider, and the patient is double-booked if they agree. In some embodiments, the highest-risk slots are flagged to allow the scheduler to estimate a timeframe for the double-booked patient to stand by. The scheduling module 206 further displays the double-book opportunity 265 for the time interval with respect to the cumulative risk 245 across the time interval. As illustrated in FIG. 7, a double book opportunity is shown for an afternoon time interval in which there are no double bookings placed, but the afternoon block displays the double-book opportunity 265 to notify a practitioner or scheduler that a double-booking opportunity is available over the interval.

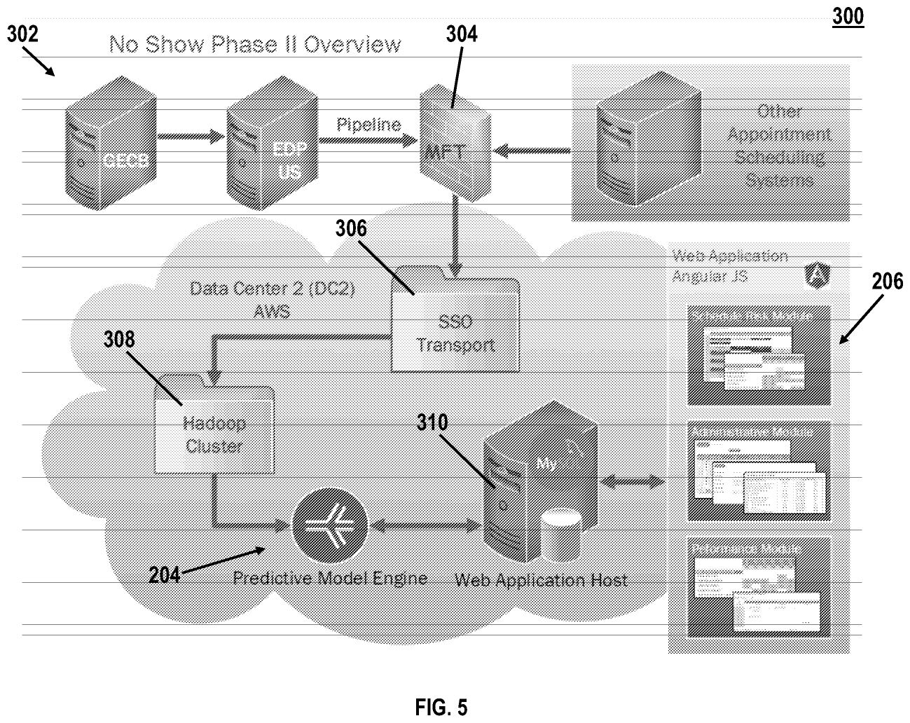

Referring to FIG. 5, a simplified diagram 300 is illustrated showing an example implementation of aspects of the predictive double booking system 200. The predictive model engine 204 of the predictive double booking system 200 receives attributes 220 for each appointment slot of a plurality of appointment slots within a time interval. In some embodiments, a Managed File Transfer (MFT) module 304 extracts the attributes 220 among other information from EHR systems 302 that include various appointment scheduling systems and EHR data for a plurality of patients. An access control module 306 is further included to prevent unauthorized entry or access to the data. In one embodiment, the access control module 306 is a single sign-on (SSO) service. In some embodiments, the data is organized at a device 110 such as cluster 308, which is in some embodiments a Hadoop cluster.

The predictive model engine 204 of the predictive double booking system 200 communicates with the cluster 308 to obtain the attributes 220 among other information from the cluster 308 and assess no-show risk across the plurality of appointment slots within a time interval as discussed above. In some embodiments, the results from the predictive model engine 204 are communicated to a web application host 310 such as SQL. In some embodiments, web application host 310 facilitates the scheduling module 206 that allows a user to double-book an additional appointment time interval based on the risk score for the time interval as determined by the predictive model engine 204.

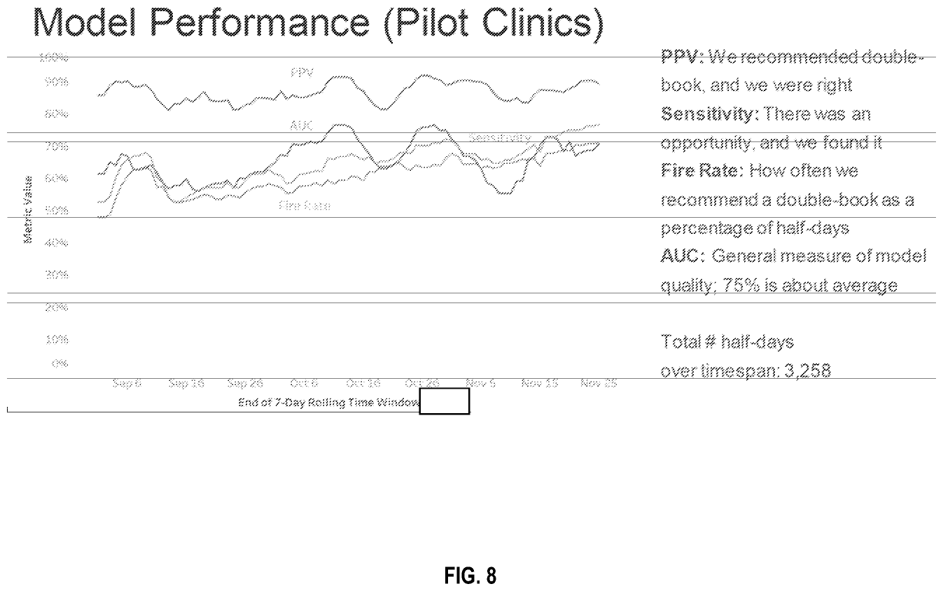

FIGS. 6 and 7 illustrate the scheduling tool 270 embodied as a web-based GUI showing a risk summary view 262 and associated schedule summary view 263 that shows risk assessments for a plurality of providers across half-day time intervals. FIG. 8 is illustrative of performance metrics over the course of a week using the predictive double-booking system 200. Table 1 illustrates a set of variable names and definitions used for the one embodiment of the predictive double-booking system 200.

TABLE 1
Variable names and definitions

| Variable | Description |
|---|---|
| AppointmentNumber | ID for the appointment (a scheduled |
| | block of time in which a patient visits |
| | with a provider in an outpatient clinic) |
| AppointmentDate | The day upon which the appointment will |
| | be held |
| AppointmentTime | The time at which the appointment will |
| | be started |
| CreateDate | The day upon which the patient booked |
| | the appointment |
| CreateTime | The time at which the patient booked |
| | the appointment |
| DurationOfAppointment | How long the patient is scheduled to |
| | visit with the provider |
| AppointmentStatusName | The original target variable. Denotes |
| | whether the patient arrived, canceled, |
| | no-showed, rescheduled, was bumped, |
| | or has an appointment in the future |
| | (pending) |
| AppointmentType | Free text describing the main reason |
| | for the visit |
| CancelDate | The day upon which the patient decided |
| | to cancel the appointment |
| CancellationReason | Categorical field classifying why the |
| | appointment was canceled |
| DateArrived | The day upon which the patient arrived |
| | for their appointment. In the vast |
| | majority of cases should be the same |
| | as “AppointmentDate” |
| TimeArrived | The time at which the patient arrived |
| | for their appointment. In the majority |
| | of cases should be before |
| | “AppointmentTime” |
| DateOfNoShow | The day upon which the patient did not |
| | arrive for their appointment with no |
| | prior indication of absence |
| DateRescheduled | The day to which the patient moved the |
| | appointment |
| DivisionName | Categorical field for the type of clinic |
| | based on the service line it primarily |
| | operates under |
| BillingLocationName | The name of the clinic. Main ID for |
| | the facility |
| BillingAreaName | Categorical field for the kind of |
| | physical location in which the clinic |
| | is situated in. Almost always ‘OFFICE’ |
| SchedulingProviderName | The name of the care provider. Main ID |
| | for the provider |
| SchedulingLocationName | The name of the specific part of the |
| | clinic in which this appointment will |
| | take place. Often 1:1 with |
| | “BillingLocationName” but not always. |
| SchedulingLocationAddress1 | The general street address for the |
| | clinic's building |
| SchedulingLocationAddress2 | The clinic's specific suite or unit |
| | at the building |
| SchedulingLocationCityState | ‘city, state’ combo in which the |
| | clinic is located |
| SchedulingLocationZipCode | The ZIP code in which the clinic is |
| | located |
| PatientDateOfBirth | The day when the patient was born. |
| | Can be used to calculate age |
| MedicalRecordNumber | ID for the patient, typically unique at |
| | the region level |
| PatientSex | Male, female or other |
| PatientMaritalStatus | Categorical field for if the patient is |
| | or was married. Different descriptions of |
| | past, present and no marriage are available |
| PatientAddress1 | The general street address for the patient |
| PatientAddress2 | The patient's specific unit, apartment |
| | or suite at their building |
| PatientCity | The city in which the patient is located |
| PatientState | The state in which the patient is located |
| PatientZip | The ZIP code in which the patient is |
| | located |
| CarrierName | The name of the patient's insurer |
| SubscriberNumber | How the insurer identifies the patient |
| PrimaryInsuranceClass | Categorical field for the name of the |
| | general insurance plan the patient is |
| | under. The general insurance company |
| | but with some nuanced detail about the |
| | plan itself. For the primary insurance. |
| PrimaryFinancialClass | Categorical field for the kind of |
| | organization the insurer is (Medicare, |
| | Medicaid, commercial, self-pay, etc.). |
| | For the primary insurance. |
| SecondaryInsuranceClass | Categorical field for the name of the |
| | general insurance plan the patient is |
| | under. The general insurance company |
| | but with some nuanced detail about the |
| | plan itself. For the secondary insurance. |
| SecondaryFinancialClass | Categorical field for the kind of |
| | organization the insurer is (Medicare, |
| | Medicaid, commercial, self-pay, etc.). |
| | For the secondary insurance. |
| TertiaryInsuranceClass | Categorical field for the name of the |
| | general insurance plan the patient is |
| | under. The general insurance company |
| | but with some nuanced detail about the |
| | plan itself. For the tertiary insurance. |
| TertiaryFinancialClass | Categorical field for the kind of |
| | organization the insurer is (Medicare, |
| | Medicaid, commercial, self-pay, etc.). |
| | For the tertiary insurance. |

The analysis itself could be expanded to many more markets, which would require some rethinking about how to curate and standardize the data. The gradient boosting version of the model could be implemented instead of the logistic regression. One could also trade out the three-rule universal model for a full version. The predictive double-booking system 200 tries to pick cutoffs that keep the PPV at each clinic at approximately 85% —as such, an automated way of adjusting cutoffs to keep this metric stable would be helpful. Techniques from Bayesian probability could be applied here, as well as traditional (and less traditional) process control algorithms. There could be a lot of value in making the recommendations more time-specific—that is, narrowing the range of time in a schedule when a no-show can be expected, or event selecting individual slots. This can be achieved by adjusting the aggregation step to look at sequential blocks of appointments that are a subset of the full half-day and that meet the predictive performance criterion.

### Feature Spaces: Selecting Predictive Factors

In some embodiments, a number of predictive features gleaned from the patient data 221, clinic data 222, and appointment data 223 can include items such as patient age, appointment lead time, and patient marital status. Other predictive features can include previous patient no-show history, a ZIP code, patient insurance class, marital status and appointment type (established patient, new patient, follow up, etc.). The predictive features which are explored will naturally differ between embodiments and should be as specific and fair to the applicable situation as possible to avoid unfair, problematic or inaccurate generalizations. Thus, a clinic classification approach is implemented for some embodiments, where the predictive double-booking system 200 can consider different predictive features for each of a plurality of clinic classifications.

In one example embodiment of the predictive double-booking system 200, some clinic classifications that are considered include Internal Medicine, Surgery, and “Nevada” (in reference to a singular outlier clinic in Nevada, given that geographical location will have its own effect on the features considered). Since the predictive double-booking system 200 performs better when using application-specific predictive features, the clinic classifications considered can vary based on the needs of the scheduling practitioner. In some embodiments, a generalized “universal” classification is included in the plurality of clinic classifications as a catch-all classification for any other clinics considered which do not fit into the three main clinic classifications. Furthermore, the number and types of clinic classifications may be altered as needed.

In the example embodiment of the predictive double-booking system 200, a prior no-show rate was empirically found to be a top predictive feature for all clinic classifications. For example, the internal medicine classification was heavily influenced by predictive features such as marital status, patient ZIP codes, and established patients in the positive direction towards higher risk. In the other direction, the internal medicine was empirically found to be less influenced by certain predictive features including insurance information, marital status, a current season or month of the year and patient ZIP codes in the negative direction towards lower risk.

The surgery clinic classification was also heavily influenced by certain predictive features such as insurance financial class, the type of appointment, and patient ZIP codes in the positive direction. The surgery clinic classification was influenced by insurance financial class, marital status, and patient ZIP codes in the negative direction.

The Nevada classification was also heavily influenced by the specific providers and one specific clinic in the positive direction; it was influenced by specific providers, clinic ZIP codes, insurance financial class, and marital status in the negative direction.

In one aspect of the predictive double-booking system 200, the clinic classifications can include large general practice clinics, medium opportunity clinics (identified via bubble plots) and outlier clinics varying in geographical location. It was found that a prior no-show rate for the patient was by far the most important predictive feature. Age and appointment lead time were also important predictive features. Marital status was determined to be less significant for these classifications.

In another aspect of the predictive double-booking system 200, clinic classifications are not relied on, and a plurality of predictive features considered include age at the time of appointment, appointment creation lead time, and marital status. These predictive features were retained as universal classification of the plurality of clinic classifications agnostic of clinic type or geography. Preliminary testing showed that patients are at a higher risk of being a no-show when the patient is 55 years old or older, the patient schedules an appointment more than 32 hours prior to the start time of the scheduled appointment, or when the patient is not married or partnered.

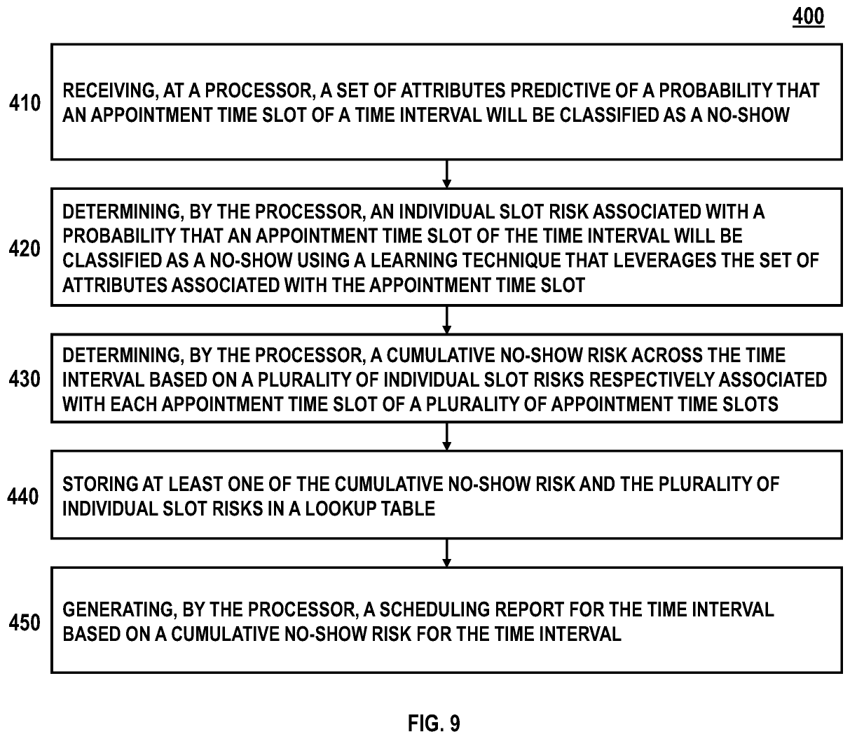

FIG. 9 illustrates an example simplified procedure 400 for determining a cumulative risk of a no-show across a time interval in accordance with the examples and processes described above. For purposes of discussion, the operations for procedure 400 are described in the context of a system such as predictive double booking system 200. However, it is appreciated that these operations may be performed by any number of systems and devices, including combinations of systems and devices in a distributed computing environment.

At block 410, the system receives at a processor a set of attributes that are predictive of a probability that an appointment time slot of a time interval will be classified as a no-show (e.g. the patient does will not attend the appointment). Such attributes can include patient attributes and data, clinic data and appointment data for each appointment-level slot within a time interval.

At block 420, the system determines an individual slot risk associated with a probability that an appointment time slot of the time interval will be classified as a no-show. In some embodiments, the individual slot risk is determined using a learning technique such as logistic regression that leverages the set of attributes associated with the appointment time slot to determine whether the particular appointment time slot is at risk for being a no-show.

At block 430, the system determines a cumulative no-show risk across the time interval based on a plurality of individual slot risks respectively associated with each appointment time slot of a plurality of appointment time slots. More specifically, the cumulative no-show risk is determined using binary probability logic. The cumulative no-show risk is representative of a probability that at least one appointment time slot across a time interval will be a no-show.

At block 440 the system stores the risk scores including the cumulative no-show risk and the plurality of individual slot risks in a lookup table for reference. At block 450, the system generates a scheduling report based on the cumulative no-show risk. In some embodiments, the scheduling report is integrated within a scheduling system to allow a practitioner to use the cumulative risk score to book an additional patient.

FIG. 10 is a schematic block diagram of an example device 500 that may be used with one or more embodiments described herein, e.g., as a component of predictive double-booking system 200 and/or as any one of devices 110 shown in FIG. 1.

Device 500 includes one or more network interfaces 510 (e.g., wired, wireless, PLC, etc.), at least one processor 520, and a memory 540 interconnected by a system bus 550, as well as a power supply 560 (e.g., battery, plug-in, etc.).

Network interface(s) 510 include the mechanical, electrical, and signaling circuitry for communicating data over the communication links coupled to communication network 105. Network interfaces 510 are configured to transmit and/or receive data using a variety of different communication protocols. As illustrated, the box representing network interfaces 510 is shown for simplicity, and it is appreciated that such interfaces may represent different types of network connections such as wireless and wired (physical) connections. Network interfaces 510 are shown separately from power supply 560; however, it is appreciated that the interfaces that support PLC protocols may communicate through power supply 560 and/or may be an integral component coupled to power supply 560.

Memory 540 includes a plurality of storage locations that are addressable by processor 520 and network interfaces 510 for storing software programs and data structures associated with the embodiments described herein. In some embodiments, device 500 may have limited memory or no memory (e.g., no memory for storage other than for programs/processes operating on the device and associated caches).

Processor 520 includes hardware elements or logic adapted to execute the software programs (e.g., instructions) and manipulate data structures 545. An operating system 542, portions of which are typically resident in memory 540 and executed by the processor, functionally organizes device 500 by, inter alia, invoking operations in support of software processes and/or services executing on the device. These software processes and/or services may include predictive double-booking process/services 544, described herein. Note that while predictive double-booking process/services 544 is illustrated in centralized memory 540, alternative embodiments provide for the process to be operated within the network interfaces 510, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the predictive double-booking process 544 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method, comprising:
accessing, at a processor, a set of attributes predictive of a probability that an appointment time slot of a time interval will be classified as a no-show;
determining, by the processor, an individual slot risk associated with a probability that the appointment time slot of the time interval will be classified as a no-show using a learning technique that leverages the set of attributes associated with the appointment time slot;
determining, by the processor, a cumulative no-show risk across the time interval based on a plurality of individual slot risks respectively associated with each appointment time slot of a plurality of appointment time slots; and
generating, by the processor, a scheduling report for the time interval based on a cumulative no-show risk for the time interval, wherein the scheduling report is configured to denote a double book opportunity for the time interval based on the cumulative no-show risk for the time interval.

2. The method of claim 1, further comprising:
applying a logistic regression learning technique to the set of attributes associated with the appointment time slot.

3. The method of claim 1, further comprising:
determining an arrival probability associated with a probability that the appointment time slot of a time interval will not be classified as a no-show based on the individual slot risk;
determining a probability that all appointment time slots of the time interval will not be classified as a no-show based on a combination of a plurality of arrival probabilities respectively associated with each appointment time slot of the plurality of appointment time slots of the time interval; and
obtaining the cumulative no-show risk that at least one appointment time slot of the time interval will be classified as a no-show across the time interval.

4. The method of claim 1, further comprising:
storing at least one of the cumulative no-show risk and the plurality of individual slot risks in a lookup table.

5. The method of claim 4, further comprising:
retrieving at least one of the cumulative no-show risk and the plurality of individual slot risks from the lookup table upon report generation.

6. The method of claim 1, wherein the set of attributes predictive of a probability that an appointment time slot of a time interval will be classified as a no-show includes at least two of patient data, clinic data, and appointment data.

7. The method of claim 1, wherein the scheduling report further includes the plurality of individual slot risk assessments associated with the plurality of appointment time slots of the time interval.

8. The method of claim 1, further comprising:
applying a gradient boosted decision tree algorithm to the set of attributes associated with the appointment time slot.

9. The method of claim 1, wherein the set of attributes is dependent upon a clinic classification, wherein the clinic classification is determined using the clinic information of the set of appointment data.

10. The method of claim 1, further comprising:
comparing the cumulative no-show risk for the time interval with a predetermined threshold value; and
identifying the double-book opportunity for the time interval based on a comparison between the cumulative no-show risk and the predetermined threshold value.

11. The method of claim 1, further comprising:
double-booking an appointment within the time interval based on the double-booking opportunity.

12. A device, comprising:
one or more network interfaces to communicate over a network;
a processor coupled to the network interfaces and adapted to execute one or more processes; and
a memory configured to store a process executable by the processor, the process, when executed, is operable to:
access a set of attributes predictive of a probability that an appointment time slot of a time interval will be classified as a no-show;
determine an individual slot risk associated with a probability that the appointment time slot of the time interval will be classified as a no-show using a learning technique that leverages the set of attributes associated with the appointment time slot;
determine a cumulative no-show risk across the time interval based on a plurality of individual slot risks respectively associated with each appointment time slot of a plurality of appointment time slots; and
generate a scheduling report for the time interval based on a cumulative no-show risk for the time interval.

13. The device of claim 12, wherein the process, when executed, is further operable to:
denote on the scheduling report a double book opportunity for the time interval based on the cumulative no-show risk for the time interval.

14. The device of claim 13, wherein the process, when executed, is further operable to:
compare the cumulative no-show risk for the time interval with a predetermined threshold value; and
identify the double book opportunity for the time interval based on a comparison between the cumulative no-show risk and the predetermined threshold value.

15. The device of claim 13, wherein the process, when executed, is further operable to:
double-book an appointment within the time interval based on the double book opportunity.

16. The device of claim 12, wherein the process, when executed, is further operable to:
apply a logistic regression learning technique to the set of attributes associated with the appointment time slot.

17. The device of claim 12, wherein the set of attributes predictive of a probability that an appointment time slot of a time interval will be classified as a no-show includes at least two of patient data, clinic data, and appointment data.

18. The method of claim 12, wherein the set of attributes is dependent upon a clinic classification, wherein the clinic classification is determined using the clinic information of the set of appointment data.

19. The device of claim 12, wherein the process, when executed, is further operable to:
determine an arrival probability associated with a probability that the appointment time slot of a time interval will not be classified as a no-show based on the individual slot risk;
determine a probability that all appointment time slots of the time interval will not be classified as a no-show based on a combination of a plurality of arrival probabilities respectively associated with each appointment time slot of the plurality of appointment time slots of the time interval; and
obtain the cumulative no-show risk that at least one appointment time slot of the time interval will be classified as a no-show across the time interval.

20. A tangible, non-transitory computer-readable medium having instructions encoded thereon the instructions, when executed by a processor, are operable to:
access a set of attributes predictive of a probability that an appointment time slot of a time interval will be classified as a no-show;
determine an individual slot risk associated with a probability that the appointment time slot of the time interval will be classified as a no-show using a learning technique that leverages the set of attributes associated with the appointment time slot;
determine a cumulative no-show risk across the time interval based on a plurality of individual slot risks respectively associated with each appointment time slot of a plurality of appointment time slots; and
generate a scheduling report for the time interval based on a cumulative no-show risk for the time interval.

21-24. (canceled)
